(19) United States
(12) Patent Application Publication
(10) Pub. No.: US 2001/0029383 A1
(43) Pub. Date: Oct. 11, 2001

(54) Connecting apparatus and method

(76) Inventor: Jan Otto Solem, Stetten (CH)

Correspondence Address: BROWDY AND NEIMARK, P.L.L.C., 624 NINTH STREET, NW, SUITE 300, WASHINGTON, DC, 20001-5303, US

(21) Appl. No.: 09/769,748

(22) Filed: Jan. 26, 2001

Related U.S. Application Data
(63) Continuation-in-part of application No. 09/228,749, filed on Jan. 12, 1999, now Pat. No. 6,179,848.

(30) Foreign Application Priority Data
Jul. 24, 1996 (SE) 9602849-3

(51) Int. Cl. A61B 17/08

(52) U.S. Cl. 606/153

(57) ABSTRACT

For connecting the end of a supplemental vessel to the side of a body vessel, which has a greater diameter than the supplemental vessel, use is made of a sleeve whose one end portion is expandable for the forming of an annular end flange. As a result, the supplemental vessel, after being passed through the sleeve in the direction of the expandable end portion and folding back at least over the expandable end portion, is formable into a collar, both sides of which enclose the annular end flange inside the supplemental vessel and internally surround an opening formed therein. The distal portion of the sleeve to be inserted into the body vessel may comprise a memory material enabling the end flange to be formed without using the balloon.

### CROSS REFERENCE TO RELATED APPLICATION

This application is a continuation-in-part of U.S. patent application Ser. No. 09/228,749 that was filed on Jul. 23, 1997.

### BACKGROUND OF THE INVENTION

1. Technical Field of the Invention

The present invention relates to the field of vascular surgery and, more specifically, to a connecting method and a connecting apparatus for vessels, especially the aorta in bypass operations on the coronary vessels. The apparatus may be used for connecting any smaller vessel like a vein, an artery or a graft of artifical material end to side of a bigger vessel like the aorta. The artificial material could be PTFE, polyurethane and Dacron. The invention might also be used for an end to side connection to other tubular organs of the human body such as big veins, biliary tracts and urogenital tracts.

2. Description of the Prior Art

An increased flow resistance in the various coronary vessels can jeopardise the oxygen supply to the cardiac muscle. In some cases an expansion of the vascular lumen is possible. If the flow of blood in a vessel is completely or practically completely blocked, the only thing to be done is to bypass the blocked portion to prevent an irreparable injury from arising. Such a bypass operation is usually effected by connecting a new vessel after the blocked point and connecting it to another blood vessel, for instance the aorta, which may give a sufficient flow of blood to the blood vessel after the blocked point.

Such a bypass operation normally requires the use of a heart-lung machine, i.e. that the heart be temporarily stopped, since the bypass operation when connecting, for instance, the two vessels involved requires the heart to be immovable. In consequence of the connecting technique employed and the use of the heart-lung machine, the operation will be relatively time-consuming and not without risk.

International Patent Application No. PCT/SE97/00804 (equivalent to U.S. patent application Ser. No. 09/192,895, filed by the present applicant) discloses a branching device, which to a considerable extent facilitates the connection of the new vessel to the coronary vessel suffering from stenosis, but this branching device is not suited for connection of the new vessel to e.g. the aorta mainly owing to the difference in size.

### SUMMARY OF THE INVENTION

The object of the present invention therefore is to provide a simple and reliable end to side connection of a smaller supplemental vessel to a bigger body vessel, for instance the aorta, which can give a sufficient flow of blood to a constricted blood vessel via a branch after the constriction. Preferably it should also be possible in this case to design the connection in a manner which makes it possible that the heart-lung machine need not be used. Most preferably it should be possible to carry out the operation by applying endoscopy.

The inventive sleeve makes it possible to create a flange at the end of a first smaller supplemental vessel. The flange can be made to extend around the inside of an opening formed in the second bigger body vessel. As a result, a relatively large overlapping can be achieved in the connecting area between the first and the second vessel, which in turn permits a highly reliable connection of the two vessels.

In a first embodiment, use is made of a balloon for expanding the end portion of the sleeve and, thus, for forming the flange. More precisely, the balloon is equipped with an inlet tube, which can be passed through the sleeve, such that the balloon itself will be positioned adjacent to the expandable end portion of the sleeve in order to accomplish, by inflation, the expansion thereof and at the same time also form the flange at and end of the first smaller vessel.

For fixing the position of the balloon during inflation thereof such that the resulting pressure acts against the expandable end portion of the sleeve, the inlet tube of the balloon suitably is made of a substantially nonelastic material.

In the preferred embodiment of the inventive apparatus, the balloon besides has a nonelastic portion, which abuts on the inlet tube and in extended state has a shape corresponding to the desired shape of the annular end flange. This ensures still more that the expandable end portion of the sleeve is affected in the correct manner for the shape of the annular flange to be correct.

In a second embodiment, use is made of a memory material for expanding the end portion of the sleeve and, thus, for forming the end flange. More precisely, the distal end portion of the sleeve comprises a memory material that will force this end portion to assume the shape of a radial extending flange when it has been introduced into the bigger vessel. The bigger vessel could be any body vessel having a greater diameter than the smaller supplemental vessel.

Generally, the inventive apparatus comprises a sleeve having proximal and distal portions, the proximal portion adapted to couple the end of the supplemental vessel to the sleeve, the distal portion expandable from a collapsed delivery configuration, adapted to pass through the opening in the wall of the body vessel, to an expanded deployed configuration, adapted to form an annual end flange within the body vessel; and a fixing element adapted to attach the annular end flange to the wall of the body vessel.

By making the distal portion of the sleeve from a memory material the distal portion can be transformed, i.e. expanded, into the annular flange as a result of the memory material unfolding and resuming the shape of an annular flange.

In order to retain the distal portion as an axial elongation of the proximal portion of the sleeve, a cap removably engaging the distal portion may be used. This cap allows for selective expansion of the distal portion by preventing expansion and unfolding of the distal portion of the sleeve. It may be manouevred by means of a wire fixed to the cap and a tube slidable on the wire.

The proximal portion of te sleeve may comprise a stent having a film cover made of PTFE, polyurethane, Dacron or the like.

According to the invention, a locking ring is advantageously used to fix the two vessels. The locking ring can coaxially receive the sleeve and be displaced along this to a position adjacent to the annular flange, overlapping portions of the second bigger vessel or mutually overlapping portions of the first smaller vessel and the second bigger vessel being positioned therebetween. With the aid of suitable fixing means, e.g. pins with barbs, which can be integrated with the locking ring and directed axially relative to the sleeve, portions of the second bigger vessel, and possibly portions of the first smaller vessel, can be fixed in a simple and reliable manner around the opening formed in the second bigger vessel.

The entire sleeve but preferably only its expandable end portion can advantageously be made of a net-like flexible material, but the end portion of the sleeve can alternatively be formed by making a plurality of axial slots from one end of the sleeve.

An inventive apparatus comprising the memory material and the cap may be used in a method according to the invention for fixing an end of a supplemental vessel to the side of a body vessel having a greater diameter than the supplemental vessel. This method comprises: providing apparatus comprising a sleeve having proximal and distal portions, the distal portion expandable from a collapsed delivery configuration to an expanded deployed configuration to form an annular end flange, and a fixing element adapted to attach the annular flange to the vessel wall; attaching the proximal portion of the sleeve to a distal portion of the supplemental vessel; forming an opening in the wall of the body vessel; inserting the distal portion of the sleeve into the body vessel in the collapsed delivery configuration; expanding the distal portion of the sleeve to the expanded deployed configuration, thereby forming the annular flange within the body vessel; and attaching the annular end flange to the body vessel with the fixing element.

### BRIEF DESCRIPTION OF THE DRAWINGS

FIG. 1 is a perspective view and shows a heart with two schematically indicated bypasses of coronary vessels each having a blocking,

FIG. 2 is a perspective view and shows one embodiment of an apparatus according to the invention, as well as parts preferably associated therewith,

FIGS. 3 and 4 are perspective views for explaining the method of mounting the embodiment of an inventive apparatus as shown in FIG. 2,

FIG. 5 is a perspective view of the inventive sleeve with an end portion expanded into an annular flange,

FIGS. 6a-c illustrate schematically the procedure when forming a flange on the inventive sleeve when this is enclosed by a vessel,

FIG. 7 is a perspective view of a first alternative embodiment of the sleeve in FIGS. 2 and 5,

FIG. 8 is a perspective view of a second alternative embodiment of the sleeve in FIGS. 2 and 5,

FIG. 9 is a perspective view of a third alternative embodiment of the sleeve in FIGS. 2 and 5,

FIG. 10 is a perspective view of a fourth alternative embodiment of the sleeve in FIGS. 2 and 5,

FIG. 11 is a perspective view of a sleeve which is combination of the sleeves shown in FIGS. 9 and 10,

FIGS. 12-15 are perspective views schematically illustrating the mounting of the sleeve shown in FIG. 8,

FIGS. 16-18 are cross-section views along lines A-A, B-B and C-C, respectively, in FIG. 12, FIG. 19 illustrates a modification of the sleeve shown in FIG. 8, and

FIGS. 20 and 21 illustrate schematically the procedure of forming a flange on the inventive sleeve when introducing this into a vessel.

### DESCRIPTION OF THE PREFERRED EMBODIMENTS

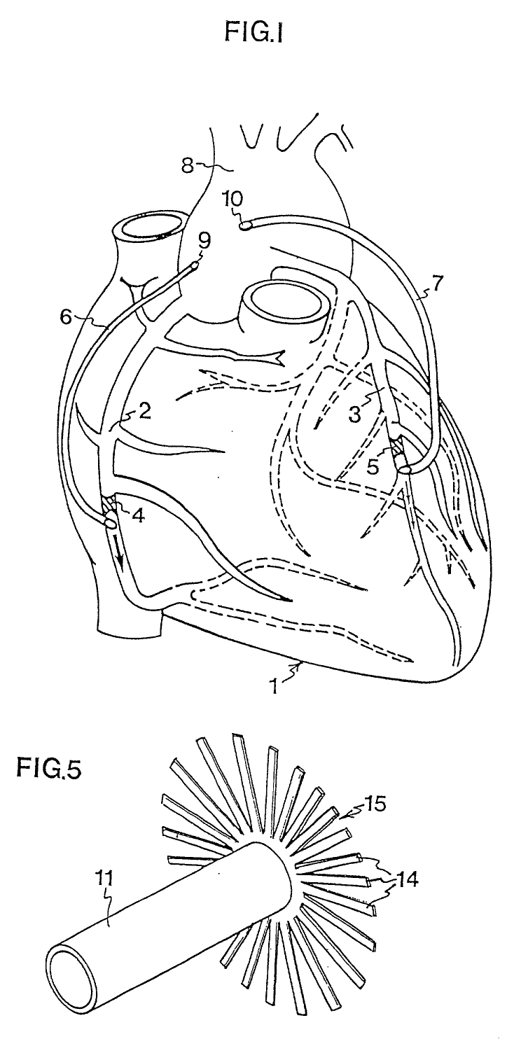

The heart 1 shown in FIG. 1 has two coronary vessels 2, 3 each having a blocking 4, 5 in the form of a stenosis or an occlusion. FIG. 1 illustrates schematically how these blockings are bypassed by means of two vessels 6, 7 which can be taken from the patient himself. More specifically, one end of the vessel 6 is connected after the blocking 4, seen in the normal direction of flow in the vessel 2, and its other end is connected to the aorta 8, such that a sufficient quantity of oxygen-rich blood will be supplied to the already blocked coronary vessel 2 after the blocking 4 therein. The same applies to the vessel 3.

For effecting the connection of the vessel 6 to the coronary vessel 2, a branching device according to International Patent Application No. PCT/SE97/00804 (and corresponding to U.S. patent application Ser. No. 09/192,895 which was filed on May 16, 1997) can be used.

The connecting apparatus according to the present invention concerns the connection of the other end 9, 10 of the vessel 6 or 7 to a vessel, e.g. the aorta 8, (i.e. a supplemental vessel to a body vessel) which thus should give a sufficient flow of blood to provide the coronary vessel 2, 3 after the blocked point 4, 5 with a sufficient supply of oxygen.

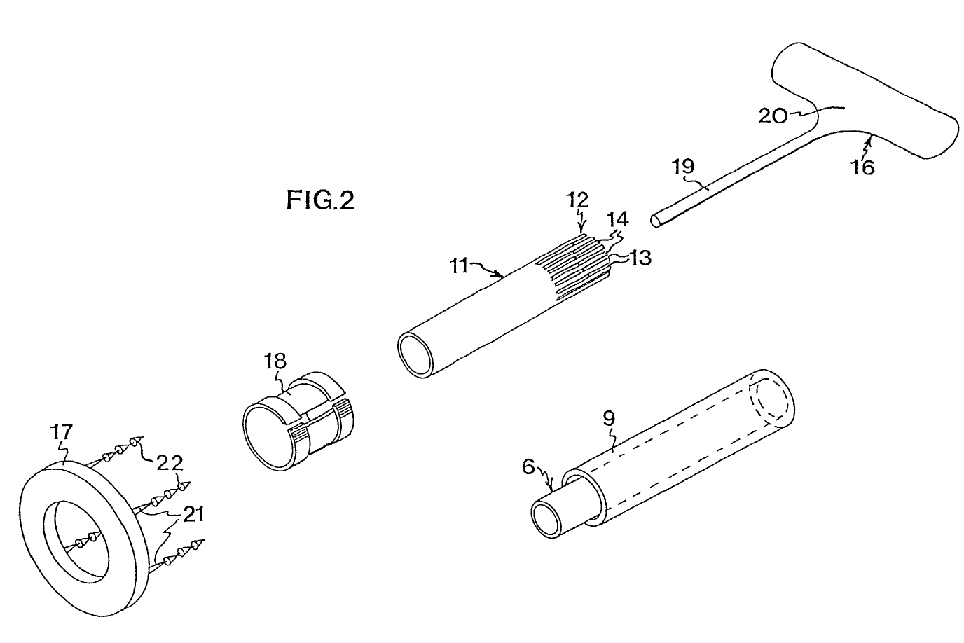

As shown in FIG. 2, the connecting apparatus according to the invention comprises a sleeve 11 of a metal or plastic that is not rejected by body tissue. The sleeve 11 comprises an end or distal portion 12 having a plurality of axial slots 13 and axially directed, intermediate ribs 14. Except for the end portion 12, the sleeve 11 is relatively rigid. The ribs 14 of the end portion 12 are also relatively rigid, but flexible outwards from their axial direction in FIG. 2 to a radial direction, as is best seen in FIG. 5. In the outwardly-flexed state, the ribs 14 form an annular radial end flange 15, as is also best seen in FIG. 5.

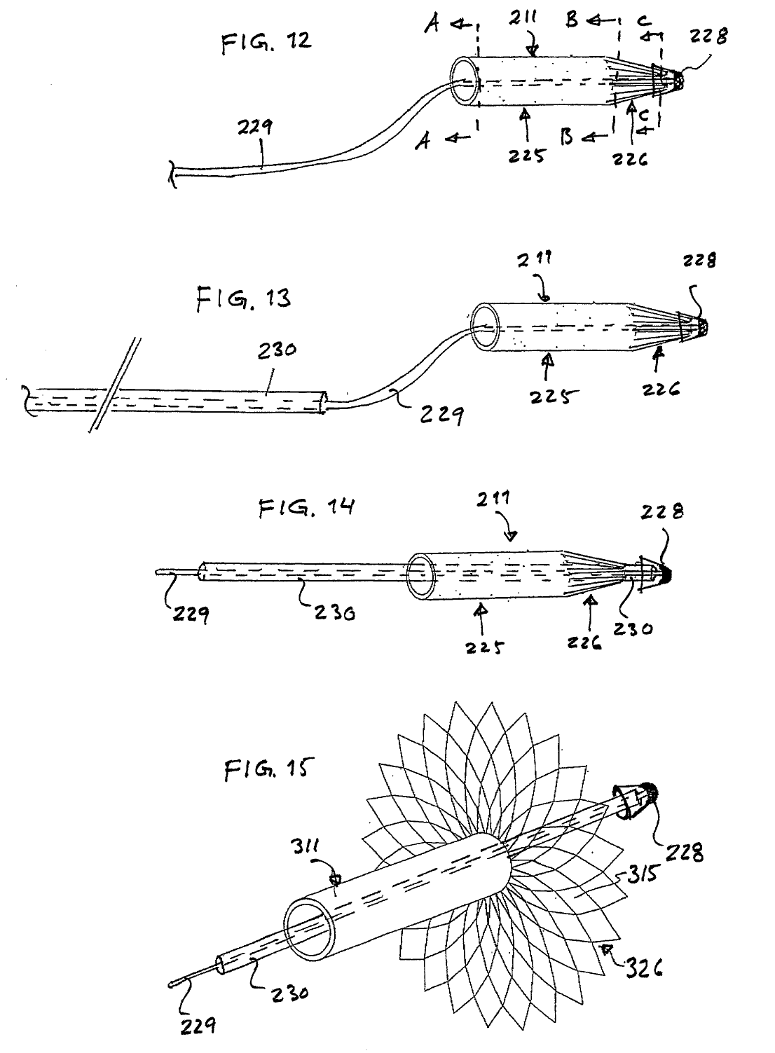

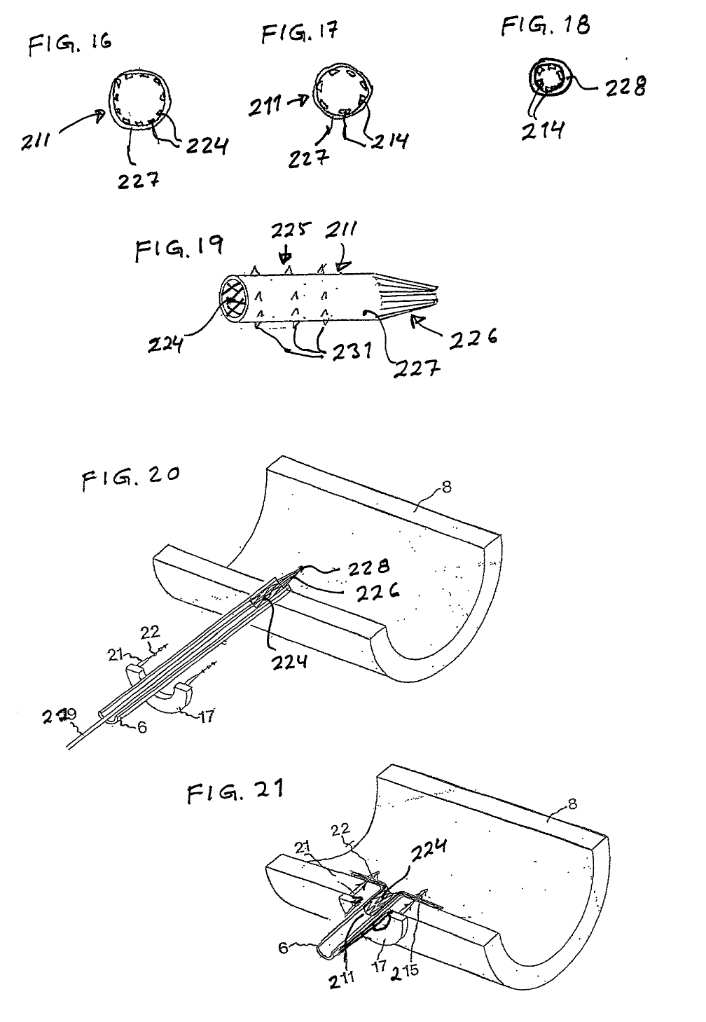

An alternative to the sleeve 11 in FIG. 5 is shown in FIG. 7. Instead of having the slots 13 and the ribs 14, the sleeve 111 in FIG. 7 is, at least at its end 112, made of a net-like flexible material, e.g. of stent type, so as to give the flange 115 of the sleeve 111 the appearance which is schematically shown in FIG. 7.

The connecting apparatus further utilises a balloon 16, a locking ring 17 and a locking sleeve 18. FIG. 2 also shows part of the first blood vessel 6 adjacent to the end 9 thereof. More precisely, the blood vessel 6 is shown in the form it gets after being passed, end 9 first, through the sleeve 11 in the direction of the end portion 12 of the sleeve and subsequently has been folded back with its outer part at least over the end portion 12 of the sleeve 11, preferably past the end portion 12.

The balloon 16 is made of an elastic material, but has an inlet tube 19 which is essentially nonelastic. The inlet tube 19 is adapted to be passed through the vessel 6, for instance after the vessel 6 has been arranged on the sleeve 11 in the manner described above. The balloon 16 may also comprise an essentially nonelastic portion 20 adjacent to the inlet tube 19 for a purpose that will be described below.

The locking ring 17 is an essentially planar ring having a plurality of axially directed pins 21, which project in the same direction from one flat side of the ring. Each pin 21 has one or more barbs 22, which ensure that the pin 21 remains once it has been passed through a material, in this case the vessel 6 and the end flange 15, as will be described in more detail below.

The locking sleeve 18 serves to safely retain on the sleeve 11 that part of the vessel 6 which has been folded back over the sleeve 11 and past the end portion 12. More specifically, the locking sleeve 18 is of such a design that it can be opened and be moved inwards laterally over that part of the vessel 6 which has been folded back over the sleeve 11, and then be clamped, such that the interiorly of the locking part positioned part of the vessel 6 is locked against the sleeve 11. Alternatively, the locking sleeve 18 can be integrally formed with the locking ring 17.

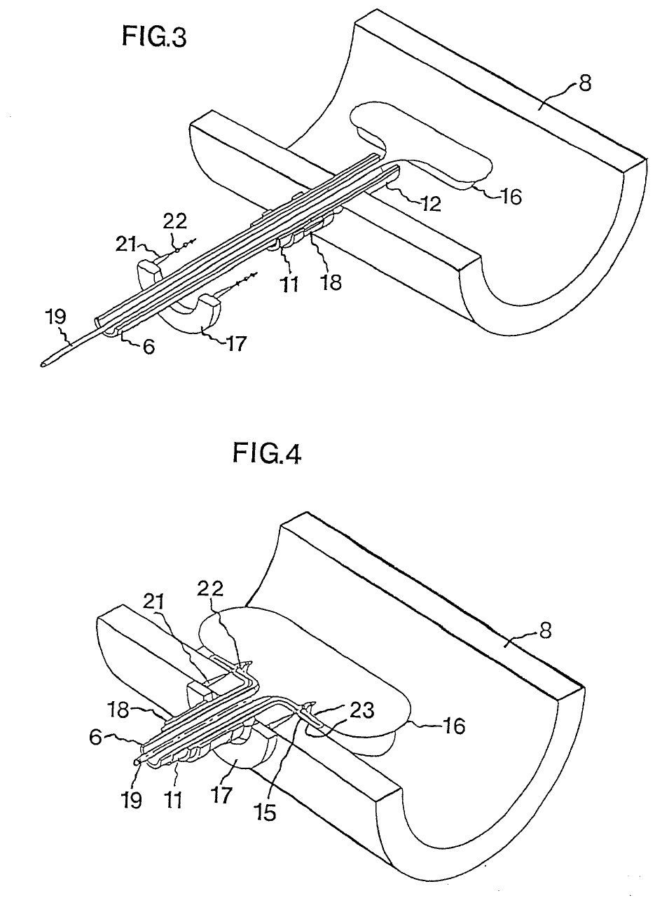

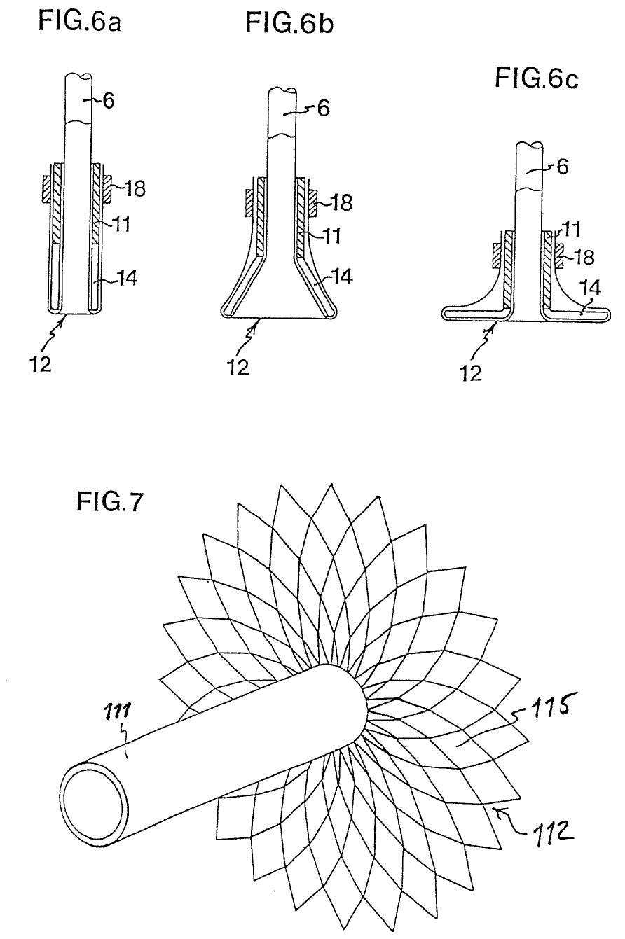

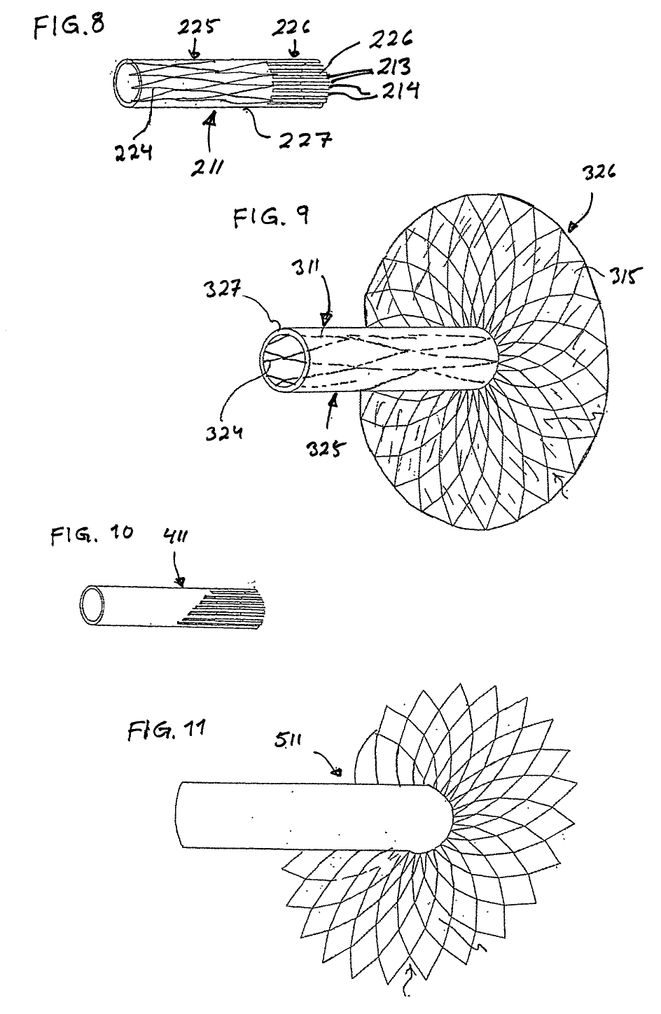

A method for connecting the vessel 6 to the vessel 8 by means of an inventive apparatus will be described below with reference to FIGS. 3 and 4.

The end 9 of the vessel 6 is first passed through the sleeve 11 and folded back over the end portion 12 and somewhat past this. The folded-back part of the vessel 6 is fixed on the sleeve 11 by means of the locking sleeve 18. The inlet tube 19 of the balloon 16 is moved through the vessel 6, and the locking ring 17 is moved inwards over the vessel 6 from the end thereof which is opposite to the balloon 16.

After an opening has been made in the wall of the vessel 8, the sleeve 11 with the vessel 6 pulled over and locked by means of the locking ring 18 and with the balloon 16 positioned within the sleeve 11 is moved so far into the vessel 8 that the end portion 12 is positioned completely inside the vessel 8. The balloon 16 is then inflated via its inlet tube 19, the balloon 16 being in such a position that the ribs 14 are bent outwards from their axial direction to a more or less radial direction. This deformation of the end portion 12 is permanent, and thus the end flange 15 is formed. The fact that the inlet tube 19 is not elastic makes it easy for the balloon 16 to affect the ribs 14 in the correct manner for the desired outwards bending thereof. The desired shape of a collar 23 formed from the vessel 6 around the end flange 15, i.e. the shape of the end flange 15, can be additionally guaranteed by the balloon portion 20 adjacent to the inlet pipe 19 also being formed essentially nonelastic.

When inflating the balloon 16, the shape of the sleeve 11 changes from the shape shown in FIG. 3 to the one in FIG. 4 (and also FIG. 5).

The change of the shape is shown in more detail in FIGS. 6a-c, where the sleeve 11 and the first vessel 6 are shown in a longitudinal cross-section, but where the balloon 16 is not included for the sake of clarity.

FIG. 6a shows the starting position, where the first vessel 6 is passed through the sleeve 11 and is folded back practically to the end of the sleeve 11 opposite the end 12, and where the locking ring 18 fixes the folded-back part of the first vessel 6 adjacent to the former end.

FIG. 6b shows the position after the expansion of the end portion 12 of the sleeve 11 has begun. The first vessel 6 will, on the inside of the sleeve 11, essentially abut on the inside of the end portion 12, while on the outside of the sleeve 11 it will extend essentially straight between the locking ring 18 and the free end of the end portion 12. Since this free end is not fixed relative to the first vessel 6, a relative movement will be possible, which is a requirement to enable expansion of the first vessel 6 without being damaged to a considerable extent.

FIG. 6c shows the final position of the expansion of the end portion 12 of the sleeve 11. Also in this case, the first vessel 6 follows the inside of the end portion 12 but does not enter the angle between the sleeve 11 and the expanded end portion 12 on the outside of the sleeve 11. This clearance between the vessel 6 and the outside of the sleeve 11 adjacent to the expanded end portion is advantageous for the necessary seal against the second vessel 8 since a pressure will thus be exerted on the inside of the edge of the opening formed in the vessel 8.

For the final fixing of the vessel 6 to the vessel 8, the locking ring 17 is moved down coaxially on the outside of the sleeve 11 towards the end flange 15, while the pins 21 penetrate at least the wall of the vessel 8 and the wall of that part of the vessel 6 which is folded back over the end portion 12 and forms one layer of the collar 23. Because of the barbs 22, the desired locking is achieved. The pins 21 can advantageously also be made to penetrate the end flange 15 and the other layer of the collar 23 and outwards into contact with the balloon 16, which, however, is so yieldable as not to be punctured by the pins 21.

Once the vessel 6 is safely connected to the vessel 8, the pressure in the balloon 16 can finally be relieved, thereby making it possible to pull out the balloon through the vessel 6 by means of the inlet tube 19.

An alternative embodiment of the sleeve 211 is illustrated in FIG. 8 and comprises a stent 224 extending along a proximal portion 225 of the sleeve 211. An end or distal portion 226 of the sleeve 211 has the same configuration as the sleeve 11 of FIG. 2, i.e. a plurality of axially extending slots 213 alternating with a corresponding plurality of ribs 214. The sleeve 211 has a continuous cover 227 made of a film of such material as PTFE, polyurethane and Dacron, at least extending over the proximal portion 225 of the sleeve 211. The continuous cover 227 may also be extended at least over a part of the end or distal portion 226 of the sleeve 211. Alternatively, the end or distal portion 226 of the sleeve 211 may be covered by flocks of PTFE or the like.

The ribs 214 consist of a memory material, e.g. a memory metal such as Nitinol, and are shown in a folded, axially extended state in FIG. 8, in which state the ribs 214 must be retained by a positive bias, as described below. When released the ribs 214 will unfold to a radially extending state and form an annular end flange, as illustrated in FIG. 5, without the need for any external force, such as that exerted by the inflating of a balloon.

A further sleeve 311 is shown in FIG. 9 in its unfolded state having an annular end flange 315. This sleeve 311 is a combination of a proximal portion 325 corresponding to the proximal portion 225 of the sleeve 211 shown in FIG. 8 and an end portion 326 corresponding to the end portion 112 of the sleeve 111 shown in FIG. 7. Thus, this end portion 326 consists of a net-like flexible material, e.g. of stent type, which however also is a memory material such that the end portion 326 automatically will assume the shape of an annular end flange 315 when released, as shown in FIG. 9. Further, the sleeve 311 has a stent 324 and also a cover 327, which extends over the proximal portion 325 and the distal end portion 326.

The proximal ends of the slots 13, 213 of each of the sleeves in FIGS. 2 and 8 lie in a common plane, which is perpendicular to the longitudinal axis of the sleeve 11, 211. The end flange formed by the unfolded ribs 14, 213 will also lie in the same plane. Thus, an end flange, which lies in a plane that is inclined towards the longitudinal axis of the sleeve, may be obtained by placing the proximal ends of the slots in that inclined plane, as illustrated by a sleeve 411 in FIG. 10. Of course, it is possible to otain the same result with an end or distal portion having the configuration illustrated in FIGS. 7 and 9, as also shown by a sleeve 511 in FIG. 11.

Referring to FIGS. 12-15, a preferred method of releasing the ribs 214 will be described. As shown in FIG. 12, a frusto-conical cap 228 is pushed over the free ends of the ribs 214 to keep them in their folded position. More precisely the distal ends of the ribs are narrowed to each other so as also to give the end or distal portion 226 of the sleeve 211 a substantially conical shape. The cap 228 is kept over-the ends of the ribs 214 by a wire 229, which is fixed to the cap 228 preferably at central point therein. The wire 229 extends through the sleeve 211 such that a positive tension applied to the wire 229 will keep the cap 228 fixed over the free distal ends of the ribs 214.

The cap 228 may have a central hole enabling the sleeve 211 to be pushed along a guide wire extending through that central hole.

In order to release the memory material of the end or distal portion 226 of the sleeve 211, a tube 230 may be pushed along the wire 229, as shown in FIG. 13, and ultimately lift the cap 228 from the distal end of the end portion 226 of the sleeve 211, as shown in FIG. 14. The memory material of the end portion 226 will consequently unfold and resume its unbiased state, as illustrated in FIG. 15 for the type of sleeve illustrated in FIG. 9. Eventually, the cap 228 and the tube 230 may. be withdrawn from the sleeve 211 together with the wire 229.

FIGS. 16-18 illustrate cross-sections of the sleeve 211 in FIGS. 8 and 12. FIG. 16 represent the cross-section view along lines A-A in FIG. 12, showing the cover 227 encircling elements of the stent 224. FIG. 17 represent the cross-section view along lines B-B in FIG. 12, showing the cover 227 encircling the ribs 214. FIG. 18 represent the cross-section view along lines C-C in FIG. 12, showing the uncovered distal tips of the ribs 214 encircled by the cap 228.

It should be noted that the cover 227 encircling the ribs 214 must be extremely flexible so as not to prevent the ribs 14 from unfolding when released from the cap 228.

Referring to FIG. 19, the stent 224 of the proximal portion 225 of the sleeve 211 may have spikes 231 extending radially outwards from the peripheral surface of the sleeve 211 and through the cover 227 in order to fix the cover 227 relative to the stent 224 and also to fix an end portion of the vessel 6 relative to the stent 224 when pushed over the proximal portion 225 of the sleeve 211.

A method of using the apparatus illustrated in FIGS. 12-15 for connecting the vessel 6 to the vessel 8 will be described below with reference to FIGS. 20 and 21.

The proximal portion 225 of the sleeve 211 is inserted and fixed in the distal end of the vessel 6, e.g. by dilating the stent 224 by means of a balloon or by allowing a self-expanding stent 224 to expand. The cap 228 is positioned over the distal tips of the ribs 214 of the distal portion 226 of the sleeve 211 and the wire 229 extends proximally from the cap 228 and through the vessel 6.

After an opening has been made in the wall of the vessel 8, a sheath (not shown) may be used for introducing the sleeve 211 so far into the vessel 8 that the distal portion 226 is positioned completely inside the vessel 8. The cap 228 is then pushed off the distal portion 226 by means of the tube 230 (not shown) whereby the ribs 214 are unfolded and form an end flange 215 approaching the inner surface of the wall of the vessel 8 around the opening therein. The cap 228 may then be withdrawn from the sleeve 211 and the vessel 6 by pulling the wire 229 proximally. Finally, the vessel 6 is fixed to the vessel 8 by means of the locking ring 17 which is moved towards the end flange 215 while the pins 21 penetrate at least the wall of the vessel 8 and the covering film 227 of the end flange 215. Because of the barbs 22, the desired locking is achieved. The pins 21 can advantageously also be made to penetrate the end flange 215.

The expert realises that several modifications of the above-described embodiments of a connecting apparatus are conceivable within the scope of the invention as defined in the appended claims. For example, the fan shape which the ribs 14 of the end flange 15 have according to FIG. 5 can also be achieved without the slots 13 by folding the material of the end portion 12 like a fan. Also, a sleeve of the type shown e.g. in FIG. 8 but comprising ribs of a non-memory material could be used in combination with a balloon. However, it is also possible to combine a balloon with a sleeve having a distal portion, which comprises a memory material.

What is claimed is:

1. Apparatus for connecting the end of a supplemental vessel to a wall of a body vessel via an opening in the wall of the body vessel, the apparatus comprising
a sleeve having proximal and distal portions, the proximal portion adapted to couple the end of the supplemental vessel to the sleeve, the distal portion expandable from a collapsed delivery configuration, adapted to pass through the opening in the wall of the body vessel, to an expanded deployed configuration, adapted to form an annual end flange within the body vessel; and
a fixing element adapted to attach the annular end flange to the wall of the body vessel.

2. The apparatus of claim 1, wherein the distal portion of the sleeve comprises a memory material providing expansion thereof to form said annular flange.

3. The apparatus of claim 2, further comprising a cap removably engaged with the distal portion of the sleeve to allow for selective expansion of the distal portion.

4. The apparatus of claim 3, wherein the cap is disposed on a distal end of the distal portion of the sleeve, a diameter of the distal portion decreasing distally towards the cap.

5. The apparatus of claim 3, further comprising a wire coupled to the cap and extending through the sleeve to facilitate engagement and disengagement of the cap with the distal portion of the sleeve.

6. The apparatus of claim 5, further comprising a tube for receiving the wire and for disengaging the cap from the distal portion of the sleeve.

7. The apparatus of claim 1, wherein the proximal portion of the sleeve comprises an expandable stent.

8. The apparatus of claim 7, wherein the stent comprises a film cover comprising an artificial material chosen from the group consisting of PTFE, polyurethane, and Dacron.

9. The apparatus of claim 7, further comprising a balloon adapted to expand the stent to couple the end of the supplemental vessel to the sleeve.

10. The apparatus of claim 2, wherein the proximal portion of the sleeve comprises an expandable stent.

11. The apparatus of claim 10, wherein the stent comprises a film cover comprising an artificial material chosen from the group consisting of PTFE, polyurethane, and Dacron.

12. The apparatus of claim 10, further comprising a balloon adapted to expand the stent to couple the end of the supplemental vessel to the sleeve.

13. The apparatus of claim 2, wherein the distal portion of the sleeve comprises a plurality of longitudinal slots extending proximally from a distal end of the distal portion.

14. The apparatus of claim 13, wherein proximal ends of the slots are disposed perpendicular to a longitudinal axis of the sleeve.

15. The apparatus of claim 13, wherein proximal ends of the slots are inclined towards a longitudinal axis of the sleeve.

16. The apparatus of claim 13, further comprising a balloon adapted to expand the distal portion of the sleeve to form the annular end flange.

17. The apparatus of claim 16, wherein the balloon has a substantially nonelastic inlet tube.

18. The apparatus of claim 16, wherein the balloon has a substantially nonelastic portion having a shape in an expanded deployed configuration of the balloon that approximates a desired shape of the annular end flange.

19. The apparatus of claim 2, wherein the distal portion is made of a net-like material.

20. The apparatus of claim 19, further comprising a balloon adapted to expand the distal portion of the sleeve to form the annular end flange.

21. The apparatus of claim 20, wherein the balloon has a substantially nonelastic inlet tube.

22. The apparatus of claim 20, wherein the balloon has a substantially nonelastic portion having a shape in an expanded deployed configuration of the balloon that approximates a desired shape of the annular end flange.

23. The apparatus of claim 22, wherein the nonelastic portion is coupled to a substantially nonelastic tube.

24. The apparatus of claim 1, wherein the fixing element comprises a locking ring disposed coaxially about the sleeve.

25. The apparatus of claim 24, wherein the fixing element comprises pins having barbs.

26. A method for fixing an end of a supplemental vessel to a wall of a body vessel, the method comprising
providing apparatus comprising a sleeve having proximal and distal portions, the distal portion expandable from a collapsed delivery configuration to an expanded deployed configuration to form an annular end flange, and a fixing element adapted to attach the annular flange to the vessel wall;
attaching the proximal portion of the sleeve to a distal portion of the supplemental vessel;
forming an opening in the wall of the body vessel;
inserting the distal portion of the sleeve into the body vessel in the collapsed delivery configuration;
expanding the distal portion of the sleeve to the expanded deployed configuration, thereby forming the annular flange within the body vessel; and
attaching the annular end flange to the body vessel with the fixing element.

27. The method of claim 26, wherein attaching the proximal portion of the sleeve to the distal portion of the supplemental vessel comprises dilating an expandable stent disposed within the proximal portion of the sleeve.

28. The method of claim 26, wherein providing apparatus comprising a sleeve having proximal and distal portions, the distal portion expandable from a collapsed delivery configuration, comprises providing the distal portion with a diameter in the collapsed delivery configuration substantially equal to a diameter of the proximal portion.

29. The method of claim 26, wherein expanding the distal portion of the sleeve comprises removing a cap from the distal portion.

30. The method of claim 26, wherein providing the fixing element comprises providing a locking ring coaxially disposed about the sleeve, and wherein attaching the annular end flange comprises inserting the locking ring into the vessel wall.

31. The method of claim 26, wherein providing a fixing element comprises providing pins having barbs, and wherein attaching the annular end flange comprises inserting the pins into the vessel wall.

32. The method of claim 26, wherein expanding the distal portion of the sleeve comprises expanding the distal portion with a balloon.
